(19) United States
(12) Patent Application Publication
(10) Pub. No.: US 2017/0232214 A1
(43) Pub. Date: Aug. 17, 2017

(54) COMPUTER AIDED MECHANICAL VENTILATION SYSTEMS AND METHODS

(71) Applicant: Children's Medical Center Corporation, Boston, MA (US)

(72) Inventor: Brian K. WALSH, Boston, MA (US)

(21) Appl. No.: 15/501,603

(22) PCT Filed: Aug. 7, 2015
(86) PCT No.: PCT/US2015/044314

§ 371 Date: Feb. 3, 2017

Related U.S. Application Data
(60) Provisional application No. 62/034,346, filed on Aug. 7, 2014.

(51) Int. Cl. A61M 16/00

(52) U.S. Cl. CPC A61M 16/0051; A61M 2205/502; A61M 2205/52; A61M 2205/3584; A61M 2230/06; A61M 2230/42; A61M 2230/432; A61M 2230/435

(57) ABSTRACT

In one embodiment, a method for categorizing a state of a subject undergoing therapy from a mechanical ventilator is disclosed. The method includes receiving, at a device, one or more cardiovascular measurements regarding the subject, a respiratory rate from the ventilator, a carbon dioxide (CO₂) measurement regarding the subject, and a tidal volume value from the ventilator. The method also includes selecting, by the device, a ventilation category based on the one or more cardiovascular measurements, the respiratory rate, the CO₂ measurement, and the tidal volume value. The method further includes retrieving, by the device, an alert that is associated with the selected ventilation category. The method additionally includes providing, by the device, the alert to a user interface device.

### RELATED APPLICATIONS

The present invention claims priority to, and the benefit under 35 U.S.C. §119(e) of U.S. provisional patent application No. 62/034,346, entitled “Computer Aided Mechanical Ventilation Systems and Methods,” filed Aug. 7, 2014. The entire content of the aforementioned patent application is incorporated herein by this reference.

### TECHNICAL FIELD

The present disclosure relates generally to systems and methods for assessing the status of a subject undergoing respiratory therapy using a mechanical ventilator. In particular, systems and methods are introduced that provide automated monitoring, categorizing, and alerting regarding the status of the subject.

### BACKGROUND

Thousands of children and adults are admitted to intensive care units each year and placed on mechanical ventilators. Despite over eighty years since the first ventilator was designed, no alternative therapies have proven to be superior. While mechanical ventilation is generally lifesaving, prolonged use of a mechanical ventilator may lead to a number of adverse conditions. Accordingly, timely liberation from mechanical ventilation may be critical to avoiding or mitigating the adverse effects of prolonged use of a mechanical ventilator.

Thus far, clinical decisions regarding the administration of a mechanical ventilator have relied heavily on the clinical judgment of the health care provider overseeing the therapy. Typically, this entails the provider making intermittent and subjective assessments of the patient's condition. For example, a health care provider may periodically assess the patient's physiological condition and make adjustments to the ventilation therapy as needed (e.g., by determining when to extubate the patient, when to adjust the ventilator's settings, etc.).

Some attempts have been made to standardize treatment protocols and guidelines when using a mechanical ventilator to treat a patient, but treatment approaches across the industry still remain widely inconsistent. In particular, studies have shown that the implementation and execution of mechanical ventilation therapy remain inconsistent and are often left to the best judgment of the health care provider.

### SUMMARY

In one embodiment, a method for categorizing a state of a subject undergoing therapy from a mechanical ventilator is disclosed. The method includes receiving, at a device, one or more cardiovascular measurements regarding the subject, a respiratory rate from the ventilator, a carbon dioxide (CO₂) measurement regarding the subject, and a tidal volume value from the ventilator. The method also includes selecting, by the device, a ventilation category based on the one or more cardiovascular measurements, the respiratory rate, the CO₂ measurement, and the tidal volume value. The method further includes retrieving, by the device, an alert that is associated with the selected ventilation category. The method additionally includes providing, by the device, the alert to a user interface device.

In some aspects, the one or more cardiovascular measurements may include at least one of: a measured heart rate of the subject, a measured blood pressure of the subject, a rate pressure product value, or a pulse pressure value. In another aspect, the one or more respiratory measurements may include at least one of the respiratory rate, the tidal volume, a fraction of inspired oxygen (FIO2), a compliance, a resistance, a CO2, or the VO2 (oxygen consumption). In another aspect, the ventilation category may be selected from a set of categories that include three or more of: an acceptable category, a hyperventilation category, a severe hyperventilation category, a hypoventilation category, a severe hypoventilation category, a tachypnea category, a severe tachypnea category, or an insufficient ventilation category. In yet a further aspect, the method may include determining that the alert is an urgent alert and the alert is provided to the user interface in response to determining that the alert is an urgent alert. The method may also include receiving, at the processor, the CO₂ measurement from an end tidal CO₂ monitor, a transcutaneous monitor, or a blood gas analyzer, in additional aspects. The method may also include receiving, at the processor, the oxygen measurement from a pulse oximeter, a transcutaneous monitor or a blood gas analyzer.

In further aspects, the method may include determining an oxygen saturation value. The oxygen saturation value may be an oxygen saturation index (OSI) or an oxygen saturation to fraction of inspired oxygen (S/F) ratio associated with the subject. The method may also include selecting an oxygenation category based on the S/F or OSI and retrieving an alert that is associated with the selected oxygenation category. The method may further include providing the alert that is associated with the oxygenation category to the user interface device. In some aspects, the oxygenation category may be selected from a set of categories that includes: an acceptable category, a mild hypoxemia category, a moderate hypoxemia category, a severe hypoxemia category, or a hyperoxia category. In yet another aspect, the method may further include analyzing a history of oxygenation categories associated with the subject to detect acute respiratory distress syndrome (ARDS) and, in response to detecting ARDS, providing an ARDS alert to the user interface device. In some aspects, the method may include evaluating a history of subject measurements and operating conditions of the ventilator to detect a potential subject condition and reporting the detected subject condition to the user interface device. The subject condition may correspond to at least one of a ventilator associated condition being present in the subject, a ventilator associated lung injury being present in the subject, the subject requiring extubation readiness testing, or the subject being ready for extubation.

In another embodiment, a system for categorizing a state of a subject undergoing therapy from a mechanical ventilator is disclosed. The system includes one or more network interfaces to communicate with a network. The system also includes a processor coupled to the one or more network interfaces and configured to execute one or more processes. The system further includes a memory configured to store a process executable by the processor. When executed, the process is operable to receive one or more cardiovascular measurements regarding the subject, a respiratory rate from the ventilator, a carbon dioxide (CO₂) measurement regarding the subject, and a tidal volume value from the ventilator. Also when executed, the process is operable to select a ventilation category based on the one or more cardiovascular measurements, the respiratory rate, the CO₂ measurement, and the tidal volume value. The process is further operable when executed to retrieve an alert that is associated with the selected ventilation category. The process is additionally operable when executed to provide the alert to a user interface device.

In a further embodiment, a non-transitory computer readable medium containing program instructions executed by a processor is disclosed. The computer readable medium includes program instructions that receive one or more cardiovascular measurements regarding the subject, a respiratory rate from the ventilator, a carbon dioxide (CO₂) measurement regarding the subject, and a tidal volume value from the ventilator. The computer readable medium also includes program instructions that select a ventilation category based on the one or more cardiovascular measurements, the respiratory rate, the CO₂ measurement, and the tidal volume value. The computer readable medium further includes program instructions that retrieve an alert that is associated with the selected ventilation category. The computer readable medium also includes program instructions that provide the alert to a user interface device.

Advantageously, the exemplary embodiments of the present invention allow the continuous monitoring of a subject undergoing respiratory therapy using a mechanical ventilator. In addition, ventilation, oxygenation, and other status categories are disclosed herein that allow a health care provider to receive alerts and alerts regarding the status of the subject undergoing the respiratory therapy.

The additional features of the present disclosure will be described infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example computer system;

FIG. 2 illustrates an example network device for categorizing a state of a subject undergoing therapy from a mechanical ventilator;

FIG. 3 illustrates example ventilation categories;

FIG. 4 illustrates example oxygenation categories;

FIG. 5 illustrates an example flow diagram of a procedure for providing an alert based on a ventilation category;

FIG. 6 illustrates an example flow diagram of a procedure for selectively providing an alert regarding a subject undergoing respiratory therapy; and

FIG. 7 illustrates an exemplary flow diagram of a procedure for selectively determining an oxygenation category.

### DESCRIPTION OF EXEMPLARY EMBODIMENTS

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

As used herein, the term “subject” is meant to refer to an animal, preferably a mammal including a non-primate (e.g., a cow, pig, horse, cat, dog, rat, mouse, etc.) and a primate (e.g., a monkey, such as a cynomolgus monkey, and a human), and more preferably a human. For example, in a hospital or other clinical setting, a subject may otherwise be referred to as a patient.

Furthermore, the control logic of the present invention may be embodied as non-transitory computer readable media on a computer readable medium containing executable program instructions executed by a processor, controller or the like.

Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

Illustratively, the techniques described herein are performed by hardware, software, and/or firmware, which may contain computer executable instructions executed by the processor 220 (or independent processor of interfaces 210) to perform functions relating to the techniques described herein, e.g., in conjunction with communication process 244. For example, the techniques herein may executed on an aggregate of servers over wireless communication protocols, and as such, may be processed by similar components understood in the art that execute those protocols, accordingly.

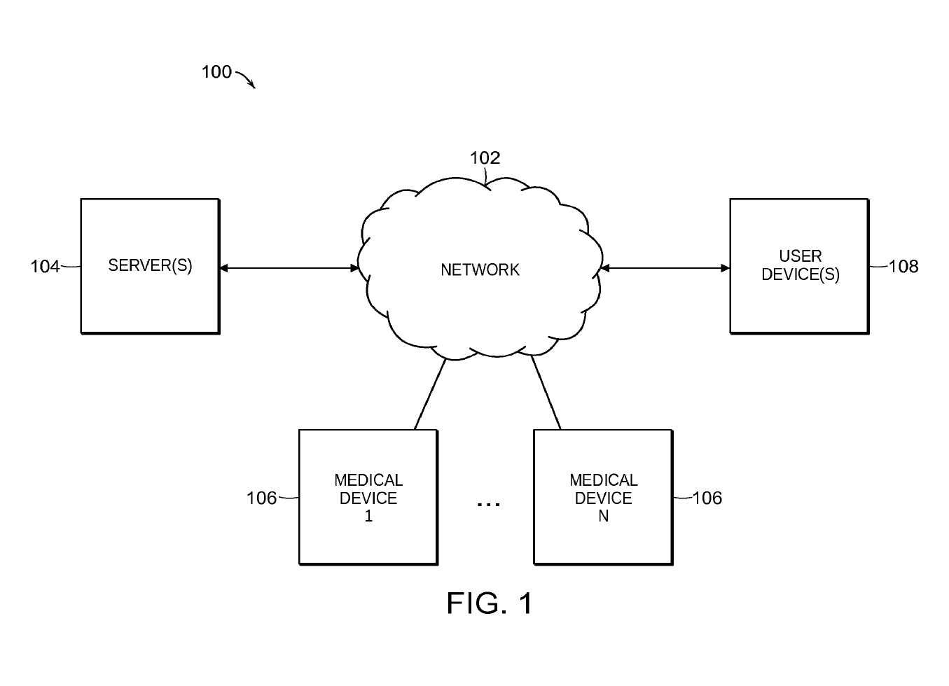

FIG. 1 is a schematic block diagram of an example computer system 100 comprising any number of user devices 108, servers 104, and/or medical devices 106 interconnected by various methods of communication which are illustratively represented as network 102. For instance, communication between the devices via network 102 may be over wired links or via a wireless communication medium (e.g., WiFi, cellular, etc.), where certain devices may be in communication with other devices based on distance, signal strength, current operational status, location, etc. Those skilled in the art will understand that any number of devices, links, etc. may be used in system 100, and that the view shown herein is for simplicity. Also, while a single network 102 is shown, network 102 may comprise any number of public or private network and/or direct connections between the devices.

According to various embodiments, medical devices 106 (e.g., a first through nth medical device) may include a ventilator (e.g., a mechanical ventilator), any sensors associated therewith (e.g., an airway pressure sensor, etc.), and any number of devices that monitor or otherwise collect/process data regarding the physiological condition of a subject undergoing therapy using the ventilator. For example, medical devices 106 may also include, but are not limited to, cardiovascular monitors (e.g., a heart rate monitor, a blood pressure monitor, etc.), any number of carbon dioxide (CO₂) sensors (e.g., an end tidal CO₂ monitor, a transcutaneous CO₂ monitor, a blood gas analyzer, etc.), any number of oxygen (O₂) sensors (e.g., a pulse oximeter (SPO2), a near infrared spectroscopy (NIRS) analyzer, a venous oximetry (SVO2) detector, a blood gas analyzer), etc.

Servers 104 may collect data from the one or more medical devices 106. The collection may be made either on a push basis (e.g., a particular medical device 106 sends data to a particular server 104 without first receiving a request to do so) or on a pull basis (e.g., the device 106 provides the data only after receiving a request for the data from server 104). The data received by servers 104 may include any data from medical devices 106 regarding the status of a subject undergoing respiratory therapy using a ventilator and/or operating parameters of the ventilator itself. Servers 104 may store the received data and may make any number of computations using the received data. For example, servers 104 may calculate any number of statistics (e.g., an average measurement, a maximal or minimal measured value, etc.) using the received data. In one embodiment, servers 104 may compute any number of values based on the received data. For example, servers 104 may compute a fraction of inspired oxygen (FIO2) value, an O₂ saturation to FIO2 ratio, etc., if not already calculated by medical devices 106 and included in the data received from medical devices 106. In another embodiment, servers 104 may compute trends using the data received from medical devices 106. For example, servers 104 may compute a moving average, estimated/predicted value, or the like based on a history of the data received from medical devices 106.

User device(s) 108 may include any device configured to convey or receive sensory input to and/or from a user. For example, user device(s) 108 may include, but are not limited to, personal computers, tablet devices, smart phones, smart watches, other wearable electronic devices, personal digital assistants (PDAs), or the like. In some case, user device(s) 108 may receive data from servers 104 and/or medical device 106. For example, servers 104 may provide a webpage interface to a particular user device 108 that displays data regarding the status of a patient to the user (e.g., current measurements or calculations, trends, alerts, etc.). In some embodiments, user device(s) 108 may be operable to provide data to servers 104 and/or to medical devices 106. For example, a web-based interface served by servers 104 may be configured to receive annotations or other manually entered data regarding the patient (e.g., lab results, demographics information, medical history information, etc.).

As would be appreciated, any of the functions described herein with respect to servers 104, medical devices 106, and user devices 108 may be performed in a distributed manner across the various devices or integrated into a singular device, in various embodiments. For example, while certain functions are described herein with respect to servers 104, these functions may alternatively be performed by any of medical devices 104 or user device(s) 108.

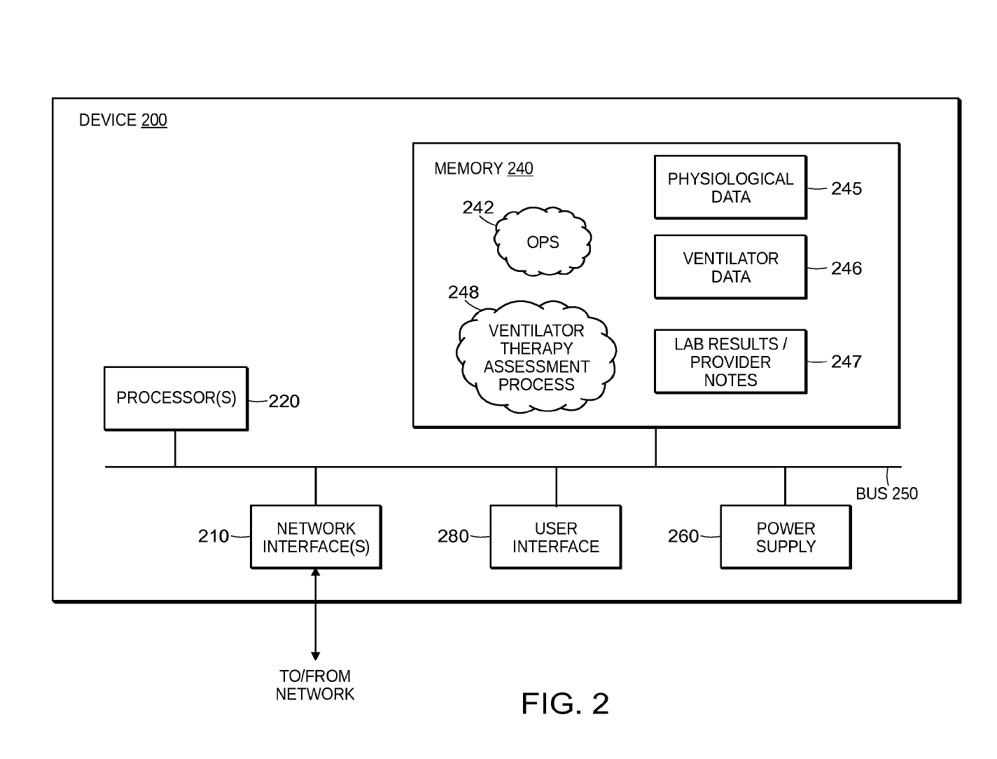

FIG. 2 is a schematic block diagram of an example device 200 that may be used with one or more embodiments described herein, e.g., as any of devices 104-108 shown in FIG. 1. The device may include one or more network interfaces 210, one or more user interfaces 280 (e.g., an electronic display, a speaker, a microphone, a keypad, etc.), at least one processor 220, and a memory 240 interconnected by a system bus 250, as well as a power supply 260 (e.g., battery, plug-in, etc.).

The network interface(s) 210 contain(s) the mechanical, electrical, and signaling circuitry for communicating data over physical and/or wireless links coupled to the network 102. The network interfaces may be configured to transmit and/or receive data using a variety of different communication protocols, including, inter alia, TCP/IP, UDP, wireless protocols (e.g., IEEE Std. 802.15.4, WiFi, Bluetooth®), Ethernet, etc. Namely, one or more interfaces may be used to communicate with the user on multiple devices and these interfaces may be synchronized using known synchronization techniques.

The memory 240 may include a plurality of storage locations that are addressable by the processor 220 and the network interfaces 210 for storing software programs and data structures associated with the exemplary embodiments described herein. As noted above, certain devices may have limited memory or no memory (e.g., no memory for storage other than for programs/processes operating on the device). The processor 220 may comprise necessary elements or logic configured to execute the software programs and manipulate the data structures, such as physiological data 245, ventilator data 246, and/or lab results provider notes and targets or goals of the therapy 247. An operating system (OPS) 242, portions of which are typically resident in memory 240 and executed by the processor, functionally organizes the device by, inter alia, invoking operations in support of software processes and/or services executing on the device. The processes and/or services may include a ventilator therapy assessment process 248, as described herein.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process). Further, while the processes have been shown separately, those skilled in the art will appreciate that processes may be routines or modules within other processes.

Ventilator therapy assessment process 248 may contain computer executable instructions executed by the processor 220 to perform the various functions described herein regarding the monitoring and analysis (e.g., categorization) of the condition of a subject undergoing respiratory therapy using a mechanical ventilator. In particular, ventilator therapy assessment process 248 may analyze physiological data 245 (e.g., received data regarding the physiological condition of the subject), ventilator data 246 (e.g., received data regarding the settings or operation of the ventilator itself), and/or data 247 that may include lab results or provider notes (e.g., digitized notes from a healthcare provider, laboratory results regarding the subject, etc.), to categorize the status of the subject. In some embodiments, ventilator therapy assessment process 248 may also contain instructions that generate alerts based on the categorized state of the subject and provide such alerts to user interface 280 or to a user interface of another device (e.g., via network 102). For example, ventilator therapy assessment process 248 may receive data from a bedside monitor, mechanical ventilator, digitized laboratory reports, radiology reports, an intravenous (IV) pump, an intracranial pressure (ICP) monitor, etc., and aggregate the data to analyze the condition of the subject. Based on the aggregated data, ventilator therapy assessment process 248 may determine that the condition of the subject falls within a particular category and, in response, provide a corresponding alert to a user interface device.

### Ventilation Categories

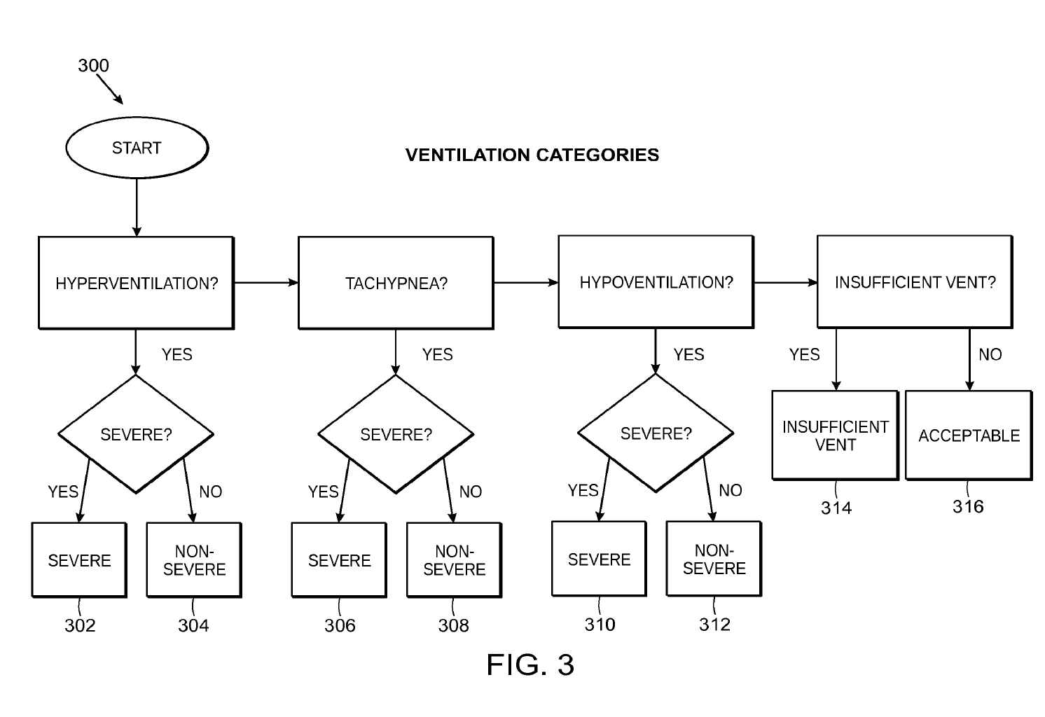

Referring now to FIG. 3, ventilator therapy assessment process 248 may categorize the status of a subject undergoing ventilator therapy based on any number of ventilation-related parameters from data 245-247. In various embodiments, the ventilation-related parameters may include data regarding the cardiovascular response of the subject. Example cardiovascular measurements may include, but are not limited to, a cardiac output (e.g., either a Fick calculation or a direct measurement), a measured heart rate (HR), a measured blood pressure (BP), a calculated rate pressure product (RPP) where RPP=HR*BP, or a calculated pulse pressure (e.g., the difference between systolic and diastolic BP measurements). In addition, ventilator therapy assessment process 248 may determine a ventilation category based in part on the, pulmonary response and/or any parameters available from the ventilator. For example, the ventilation category may be based in part on a lung compliance measurement, a lung resistance measurement, a lung elastance measurement, a minute ventilation value, a flow value, a volumetric CO₂ (VCO2) measurement, a mean airway pressure, a tidal volume (Vt) value from the ventilator, an end tidal CO₂ (ETCO₂) measurement, a respiratory rate (RR), a flow rate, a circuit pressure, a calculated measurement (e.g., ventilation index (Vd/Vt) elimination over one minute), combinations thereof, or any other calculation or value available regarding the status of the subject and/or ventilator.

As shown, ventilator therapy assessment process 248 may maintain one or more target values or acceptable ranges for each of these parameters. In one embodiment, the target values or ranges may be predefined. For example, ventilator therapy assessment process 248 may set default target values or ranges based on the age, gender, medical records, etc. of the individual subject. In another embodiment, the target values may be set manually by a health care provider (e.g., via user device 108), either by overriding any default values/ranges or by entering in new targets as the provider sees fit.

In one embodiment, ventilator therapy assessment process 248 may continuously monitor and analyze the ventilation-related parameters to categorize the status of the patient using ventilation categories 300 as follows:

TABLE 1

| Ventilation Category | Criteria |
|---|---|
| Hyperventilation 302 | CO₂ < target |
| Severe Hyperventilation 304 | CO₂ < target, |
| | HR < or > target |
| Severe Tachypnea 306 | RR > target, |
| | HR > target, |
| | or > or < |
| | BP target |
| Tachypnea 308 | RR > target |
| Hypoventilation 310 | ETCO₂ > target |
| Severe Hypoventilation 312 | ETCO₂ > target, |
| | or < |
| | HR > target, or |
| | or < |
| | BP > target |
| Insufficient Vent 314 | ETCO₂ > target, |
| | Vt < target, |
| | or < |
| | HR > target, or |
| | or < |
| | BP > target |
| Acceptable Ventilation 316 | All ventilation-related parameters within |
| | targets |
| Bradypnea | RR < target |
| Severe Bradypnea | RR < target |
| | And HR > or < |
| | Target or |
| | BR > or < target |

As shown in Table 1 above, the ventilation-related status of the subject may be categorized into a number of different categories. For example, if all of the ventilation-related parameters are within their target ranges, the status of the subject may be categorized as “Acceptable.” However, if the RR of the subject is above a target value, the subject may be categorized as experiencing “Tachypnea,” i.e., the subject is breathing too rapidly. If additional parameters are also outside of their acceptable range, the patient's condition may instead be categorized as “Severe Tachypnea.” The “Insufficient Ventilation” category may be applied if the tidal volume is below target, the ETCO₂ is above target, and/or the subject's HR is above target. Hyperventilation and hypoventilation conditions (e.g., too much ventilation and too little ventilation, respectively) each may be split, to distinguish severe cases of each condition. For example, ventilator therapy assessment process 248 may apply the “Hyperventilation” category if the ETCO₂ value is below a target value. However, if the subject's respiratory rate is above a target value, the tidal volume is above target, and/or the subject's heart rate is outside of the acceptable range, ventilator therapy assessment process 248 may instead apply the “Severe Hyperventilation” category. Ventilator therapy assessment process 248 may also apply the “Hypoventilation” or “Severe Hypoventilation” categories in a similar manner. For example, ventilator therapy assessment process 248 may apply the “Hypoventilation” category if the ETCO2 value is above target or the “Severe Hypoventilation” category if, in addition, the subject's heart rate is outside of the acceptable range, the tidal volume is below target, and/or the subject's respiratory rate is below target.

### Oxygenation Categories

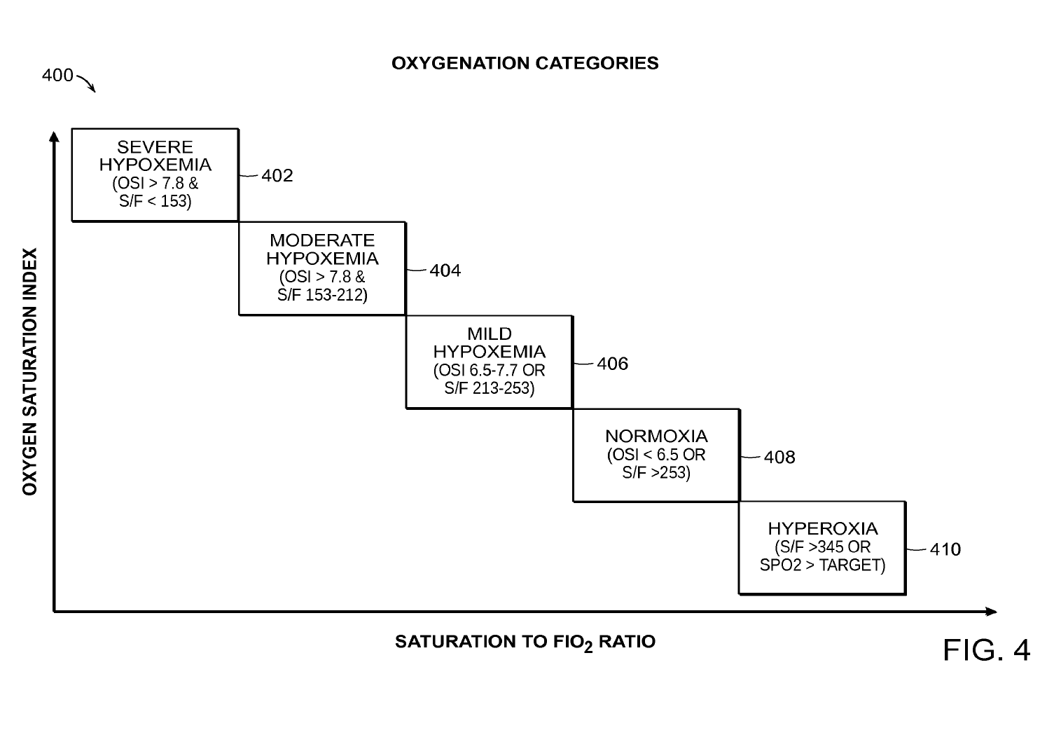

Referring now to FIG. 4, ventilator therapy assessment process 248 may categorize the status of a subject undergoing ventilator therapy based on a number of oxygenation-related parameters. Notably, ventilator therapy assessment process 248 may apply any of oxygenation categories 400 in addition to, or in lieu of, ventilation categories 300 shown in FIG. 4. In various embodiments, the oxygenation-related parameters may include a calculated ratio of the oxygen saturation (SpO2) of the subject to the fraction of inspired oxygen (FIO2), also known as an S/F ratio (i.e., S/F=SpO₂_{^{−}}/FIO2). The oxygenation-parameters may also include an oxygen saturation index (OSI) calculated as the mean airway pressure (MAP)*FIO2/SpO₂

In one embodiment, ventilator therapy assessment process 248 may continuously monitor and analyze the oxygenation-related parameters to categorize the status of the patient using oxygenation categories 400 as follows:

TABLE 2

| Oxygenation Category | Criteria |
|---|---|
| Severe Hypoxemia 402 | S/F ratio < 153 and OSI > 7.8 for > 1 hours |
| | and HR > or < target |
| Moderate Hypoxemia | S/F ratio 153-212 and OSI > 7.8 for > 2 hours |
| 404 | |
| Mild Hypoxemia 406 | S/F ratio 213-253 or OSI 6.5-7.7 for > 2 hours |
| Normoxia 408 | S/F ratio > 253 and/or OSI < 6.5 |
| Hyperoxia 410 | 0₂ > 30% with 0₂ saturation around 100% |
| | S/F ratio > 445 < 466 |

As shown above in Table 2, ventilator therapy assessment process 248 may apply the “Normoxia” (e.g., an acceptable categorization) to the subject if the S/F ratio is above a threshold value or the OSI is below a threshold value. In one illustrative embodiment, an S/F ratio greater than 253 or an OSI less than 6.5 may be categorized as normal. According to various embodiments, a hypoxemia condition (e.g., the subject is exhibiting a low amount of oxygen in his or her blood) may be divided into three categories, based on the oxygenation-related parameters: a “Mild Hypoxemia” category, a “Moderate Hypoxemia” category, and a “Severe Hypoxemia” category. Notably, ventilator therapy assessment process 248 may apply any of the hypoxemia categories only after a certain amount of time has passed (e.g., process 248 may determine whether the subject's OSI and S/F ratio are within certain ranges for a period of time, such as more than one or two hours). In some embodiments, ventilator therapy assessment process 248 may also use a “Hyperoxia” category based on the O₂ saturation of the subject and the amount of supplied O₂. Such a category may be of particular use in neonatal settings where hyperoxia is of particular concern. As will be appreciated, the target ranges/thresholds shown above in Table 2 and in FIG. 4 are exemplary only and alternate values may be set either by default or manually by a health care provider via user device 108.

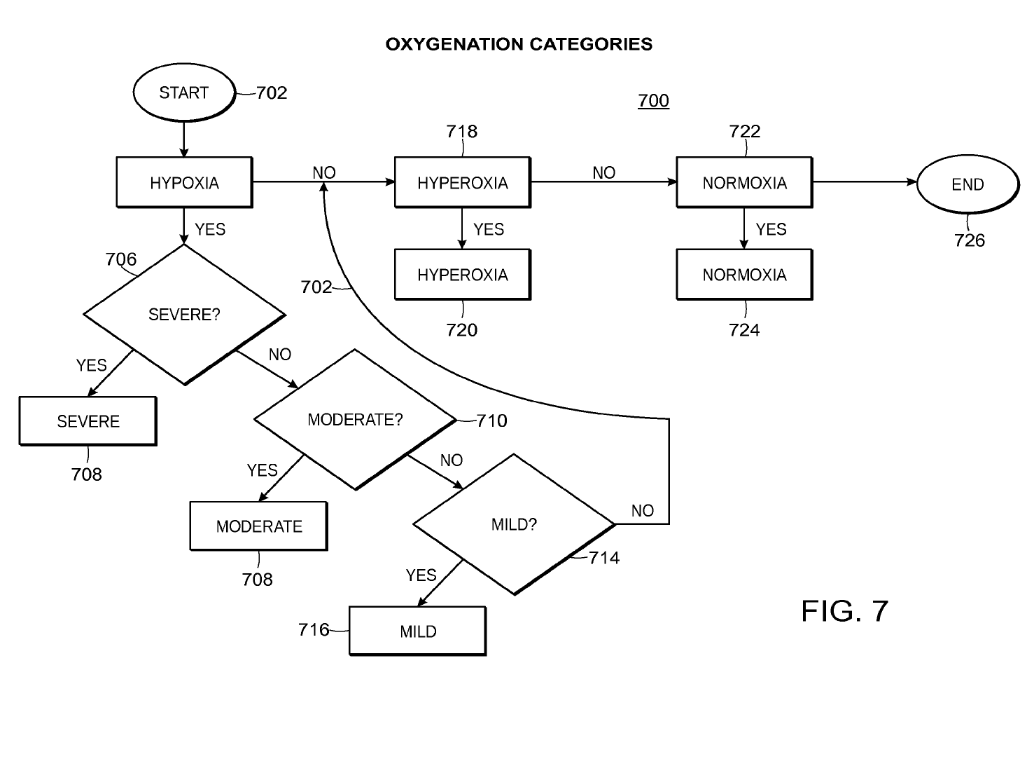

As further shown in FIG. 7, an exemplary flow diagram for providing oxygenation categorizes for a subject undergoing a ventilator therapy assessment process based on a number of oxygenation-related parameters is shown, according to various embodiments. For example, procedure 700 may be performed, in various embodiments by a device 200 executing ventilator therapy assessment process 248 or by a plurality of devices in a distributed arrangement. Procedure 700 begins at step 702 and continues on to step 704 where a determination may be made of the status of the patient with respect to the hypoxia oxygenation category. If the oxygenation criteria are satisfied then the process continues on to step 706 where the severity may be determined. If the condition is severe then the process continues on to step 708 and the oxygenation categorization of severe hypoxia may be provided as disclosed in Table 2.

At step 706, if the patient is not within the severe category the process proceeds to step 710 and an evaluation may be made to determine if the patient falls within the moderate criteria range as disclosed in Table 2. If yes, then the process proceeds to step 712 and the oxygen categorization of moderate hypoxia may be provided. If the criteria for moderate hypoxia is not satisfied, then the process proceeds on to step 714 to determine if the criteria for mild hypoxia is fulfilled as disclosed in Table 2. If the criteria is satisfied, the process proceeds on to step 716 and the oxygenation categorization of mild hypoxia is assigned.

In cases that do not meet criteria for hypoxia at step 704 or mild hypoxia at step 714, the process may continue on to step 718 to determine if the criteria for hyperoxia is fulfilled as disclosed in Table 2. If the criteria is fulfilled then the process continues on to step 720 and an oxygenation categorization of hyperoxia is assigned. Additionally, if the criteria of hyperoxia is not satisfied at step 718 the process continues on to step 722 where the criteria for normoxia is evaluated. If the criteria, as disclosed in Table 2, is fulfilled then the process continues on to step 724 and the oxygenation categorization of normoxia is assigned. If the criteria is not met then the process termination at step 726.

### Additional Categorizations

In addition to the ventilation and oxygenation categories described above, ventilator therapy assessment process 248 may also assess the physiological and ventilator data 245, 246, to identify a number of specific conditions of a subject undergoing ventilator therapy. In various embodiments, these conditions may include, but are not limited to, acute respiratory distress syndrome (ARDS), ventilator associated lung injury (VALI), the subject requiring extubation readiness testing (ERT), a ventilator associated condition (VAC) as defined by the U.S. Center for Disease Control (CDC), or the subject being ready for extubation. These categories and their corresponding criteria are shown below, according to various embodiments:

TABLE 3

| Additional Category | Criteria |
|---|---|
| ARDS | Mild Hypoxemia for > 6 hours or |
| | Moderate Hypoxemia for > 4 hours or |
| | Severe Hypoxemia for > 2 hours |
| | AND one or more |
| | radiology report |
| | consistent with low lung volumes, bilateral, |
| | infiltrates, or |
| | poor dynamic or static |
| | compliance (estimated at < 0.6 mL |
| | //cmH2O/Kg) or |
| | poor CO2 elimination |
| | fraction or |
| | an increase ventilation |
| | deadspace to tidal volume (VD/VT) > 0.7 |
| VALI | Plateau pressure (Plat) > 30 cm H₂O or |
| Stress = Pressure (force) | peak inspiratory pressure (PIP) > 32 cm |
| Strain = Volume | H₂O; |
| (response) | Hyperoxia; |
| | Vt > 8 mL/Kg or Vt < 4 ml/Kg; or |
| | positive end expiratory pressure (PEEP) |
| | not according to PEEP/FIO2 chart. |
| | Increases in PEEP or PIP that do not |
| | improve oxygenation (S/F, OSI), or |
| | compliance or ventilation (MV, ETCO2, |
| | RR) and worsen cardiac indices (e.g., HR, |
| | BP, cardiac output, pulse pressure, rate |
| | pressure product). |
| ERT | Spontaneous minute ventilation (MV) > |
| | when FIO2 < .50 and |
| | PEEP < 8 for > 2 hours consecutive |
| Extubation | All Spont. RR > when FIO2 < .50, PEEP < 6 |
| | for > 1 hours, and PIP <16, S/F > 253, |
| | and OSI < 6.5 |
| VAC | Following two days of stable or decreasing |
| | PEEP and FIO2, at least one of the |
| | indicators of worsening oxygenation |
| | occurs: |
| | 1.) >3 cm H₂O of PEEP for 2 days, or |
| | 2) >20% (20 points) increase in FIO2, |
| | sustained for 2 days. |

As would be appreciated, the above additional categories in Table 3 may use different thresholds based on the individual subject or set manually by a health care provider, according to various other embodiments.

As shown above in Table 3, ventilator therapy assessment process 248 may apply an “ARDS” category based on the amount of time the subject has been categorized in any of the hypoxemia categories. The VALI category may be based on one or more factors such as FIO2/PEEP measurements. In particular, the National Institute of Health Heart, Lung, and Blood Institute (NHLBI) ARDS Network (ARDSnet) has promulgated a low FIO2/high PEEP chart and a high FIO2/low PEEP chart as follows:

### Higher FIO2/Lower PEEP:

TABLE 4

| | | | | | | | | | | | | | | |
|---|---|---|---|---|---|---|---|---|---|---|---|---|---|---|
| FIO2 | 0.3 | 0.4 | 0.4 | 0.5 | 0.5 | 0.6 | 0.7 | 0.7 | 0.7 | 0.8 | 0.9 | 0.9 | 0.9 | 1.0 |
| PEEP | 5 | 5 | 8 | 8 | 10 | 10 | 10 | 12 | 14 | 14 | 14 | 16 | 18 | 18-24 |

### Lower FIO2/Higher PEEP:

TABLE 5

| | | | | | | | | | | | | | | |
|---|---|---|---|---|---|---|---|---|---|---|---|---|---|---|
| FIO2 | 0.3 | 0.3 | 0.3 | 0.3 | 0.3 | 0.4 | 0.4 | 0.5 | 0.5 | 0.5-0.8 | 0.8 | 0.9 | 1.0 | 1.0 |
| PEEP | 5 | 8 | 10 | 12 | 14 | 14 | 16 | 16 | 18 | 20 | 22 | 22 | 22 | 24 |

PEEP escalation will occur according to ARDSnet low FIO2/PEEP chart. Weaning recommendations will occur according to the ARDSnet high FIO2/PEEP chart. Accordingly, ventilator therapy assessment process 248 may apply the “VALI” category, if the PEEP is not in accordance with the appropriate FIO2/PEEP chart.

### Category Alerts/Recommendations

According to various embodiments, ventilator therapy assessment process 248 may retrieve and provide a category alert to a user interface device (e.g., an electronic display, a speaker, etc.) based on a ventilation, oxygenation, combination of ventilation and oxygen or additional category, as described above. In various embodiments, an alert may be a best practice alert that notifies a health care provider of a determined category and recommends a course of action. For example, if ventilator therapy assessment process 248 applies a “Severe Hypoventilation” category to the subject, ventilator therapy assessment process 248 may provide an alert to the health care provider that recommends increasing the respiratory rate of the subject.

Category alerts used by ventilator therapy assessment process 248 may also be associated with different notification times. For example, certain category alerts may be deemed urgent any may be provided to the health care provider immediately. Non-urgent alerts, however, may be stored by ventilator therapy assessment process 248 for future review by the health care provider. For example, the alert for “Mild Hypoxemia” may only be provided to the user interface device in response to a request for any alerts (e.g., as opposed to being pushed to the user interface device ventilator therapy assessment process 248 immediately on detection of the category). In some embodiments, certain types of alerts may be pushed by ventilator therapy assessment process 248 periodically or only after expiration of a timer. For example, an alert for the “Moderate Hypoxemia” category may be pushed to the user interface device once every six hours.

In one embodiment, ventilator therapy assessment process 248 may provide the following category alerts/recommendations based on practitioner or hospital established standard of practice:

TABLE 6

| | When to Send | |
|---|---|---|
| Category | Alert | Recommendation |
| Acceptable Ventilation | Never | None |
| Tachypnea | On Request | Increase Vt to upper tidal |
| | | volume or peak/plateau pressure |
| | | target value. |
| Severe Tachypnea + | Urgent/ | Increase Vt if rapid shallow |
| Hypoxemia | Immediately | breathing (high levels of |
| | | deadspace - VD/VT ratio) or |
| | | adjust PEEP if low FRC and |
| | | outside of PEEP/FIO2 chart is |
| | | suspected. |
| Severe Tachypnea without | Urgent/ | Improve CO2 elimination |
| Hypoxemia | Immediately | (VCO2 per Kg). |
| Insufficient Vent | Urgent/ | Increase Vt to within target |
| | Immediately | range. |
| Hypoventilation | On Request | Evaluate subject for |
| | | conditions that could lead to |
| | | high CO₂ production (e.g., |
| | | seizures, fever, and sepsis). |
| | | Consider adjusting ventilator |
| | | parameters to compensate for |
| | | high CO₂. Increase MV within |
| | | Vt or RR target ranges. |
| | | “CAMV Recommends xx” |
| | | based on desired PCO2 equation |
| | | as follows, but will not |
| | | recommend higher than targets: |
| | | Desired RR = [(actual RR) |
| | | (actual CO2)]/desired CO2 |
| | | Desired VT − [(actual |
| | | CO2)(actual VT)]/desired CO2 |
| Severe Hypoventilation | Urgent/ | Increase RR to within target |
| | Immediately | range. |
| | | “CAMV Recommends xx” |
| | | based on desired PCO2 equation |
| | | as follows, but will not |
| | | recommend higher than targets: |
| | | Desired RR = [(actual RR) |
| | | (actual CO2)]/desired CO2 |
| Hyperventilation | Urgent/ | Evaluate ventilator for |
| | Immediately | mechanical causes (auto trigger) |
| | | Lower MV within Vt or RR |
| | | target ranges and according to |
| | | “CAMV Recommends xx” |
| | | based on desired PCO2 equation |
| | | as follows, but will not |
| | | recommend lower than targets: |
| | | Desired RR = [(actual RR) |
| | | (actual CO2)]/desired CO2 |
| | | Desired VT − [(actual |
| | | CO2)(actual VT)]/desired CO2 |
| Severe Hyperventilation | Urgent/ | Consider metabolic, |
| | Immediately | neurological causes or |
| | | withdrawal of sedation. |
| | | Lower MV within Vt or RR |
| | | target ranges and according to |
| | | “CAMV Recommends xx” |
| | | based on desired PCO2 equation |
| | | as follows, but will not |
| | | recommend lower than targets: |
| | | Desired RR = [(actual RR) |
| | | (actual CO2)]/desired CO2 |
| | | Desired VT − [(actual |
| | | CO2)(actual VT)]/desired CO2 |
| Normoxia | Never | None |
| Mild Hypoxemia | On Demand | Adjust customizable PEEP |
| | | according to the low ARDSnet |
| | | FIO2/PEEP chart for |
| | | escalation. |
| | | OR (Clinician Choice) |
| | | Adjust customizable PEEP |
| | | according to the high ARDSnet |
| | | FIO2/PEEP chart for |
| | | escalation. |
| | | “CAMV recommends xx” the |
| | | PEEP level according to FIO2. |
| Moderate Hypoxemia | Urgent/Every 6 | Customizable PEEP according |
| | Hours until resolved | to the low ARDSnet |
| | | FIO2/PEEP chart for |
| | | escalation. |
| | | Customizable PEEP according |
| | | to the high ARDSnet |
| | | FIO2/PEEP chart for |
| | | escalation. |
| | | “CAMV recommends xx” the |
| | | PEEP level according to FIO2. |
| Severe Hypoxemia | Urgent/Every 4 | Adjust customizable PEEP |
| | Hours until resolved | according to the high ARDSnet |
| | | FIO2/PEEP PEEP chart for |
| | | escalation. Recruitment |
| | | maneuvers or prone position. |
| | | “CAMV recommends xx” the |
| | | PEEP level according to FIO2. |
| Hyperoxia | Urgent/ | Lower O₂ or PEEP level if |
| | Immediately | setting is above the |
| | | customizable FIO2/PEEP chart |
| | | “CAMV recommends xx” an |
| | | FIO2 according to desired |
| | | FIO2 equation. |
| | | Desired FIO2 = [(actual |
| | | FIO2)(desired PaO2 or SpO2)]/ |
| | | actual PaO2 or SpO2 |
| ARDS | Urgent/Every 6 | Consider implementing |
| | Hours until resolved | ARDS protocol and lower |
| | | tidal volume to 6 mL/Kg |
| | | and PEEP according to |
| | | FIO2/PEEP chart. Avoid |
| | | PIP or Plateau pressures |
| | | above 30 cmH2O. If |
| | | persistent consider alternate |
| | | therapies such as |
| | | recruitment maneuvers, |
| | | high frequency oscillatory |
| | | ventilation (HFOV), airway |
| | | pressure release ventilation |
| | | (APRV), or extracorporeal |
| | | membrane oxygenation |
| | | (ECMO). |
| VALI, Plat or PIP >30 cm | Urgent - After 2 | Consider lowering Vt if Vt > |
| H₂O | Continuous | lower |
| (barotrauma) | Hours of | bound (e.g., 4 ml/Kg). |
| Stress | Triggering | Consider alternate therapies if |
| | or | @ Vt lower bound. |
| | On Demand - | |
| | After 2 Hours of | |
| | Intermittent | |
| | Triggering | |
| VALI, Vt > upper bound | Urgent - After 2 | Consider lowering Vt. |
| (e.g., >8 ml/Kg) | Continuous | |
| (volutrauma) | Hours of | |
| Strain | Triggering | |
| | or | |
| | On Demand - | |
| | After 2 Hours of | |
| | Intermittent | |
| | Triggering | |
| VALI, FIO2 too high for | Urgent - After 2 | Consider increasing PEEP if |
| PEEP level | Continuous Hours of | low FRC is a concern. Consider |
| Strain | Triggering | pulmonary hypertension or |
| | or | Right heart failure if low FRC is |
| | On Demand - | not present. |
| | After 2 Hours of | |
| | Intermittent | |
| | Triggering | |
| VALI, FIO2 too low for | Urgent/After 2 | Consider lowering PEEP. |
| PEEP level | Continuous | CAMV recommend decreasing |
| | Hours of | by 1-2 cmH2O per event to a |
| | Triggering | minimum of 4 cmH2O. |
| Extubation Readiness | Urgent/Every 6 | Consider spontaneous breathing |
| Assessment | Hours | trial. |
| Extubation | Urgent/Every 6 | Consider Extubation. |
| | Hours | |
| VAC | Urgent/Once a | Consider further evaluation for |
| | Day | ventilator-associated event |
| | | (VAE). |

As shown above in Table 6, ventilator therapy assessment process 248 may retrieve and provide any number of category alerts to a user interface device. In some embodiments, a particular category may be associated with different alerts, depending on the conditions that triggered the category. For example, different parameters that trigger the VALI category may indicate different types of trauma and ventilator therapy assessment process 248 may provide different alerts, accordingly.

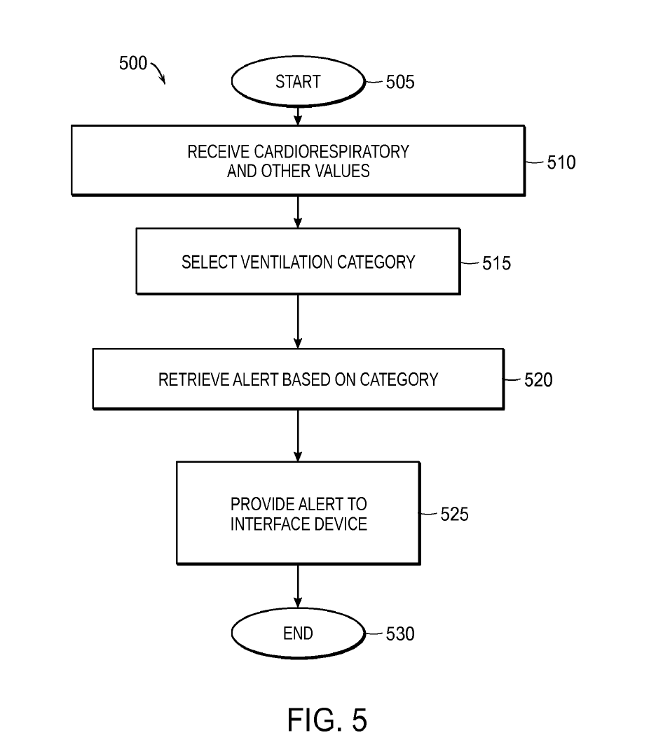

Referring now to FIG. 5, an example flow diagram of a procedure 500 for providing an alert based on a ventilation category is shown, according to various embodiments. Procedure 500 may be performed, in various embodiments, by device 200 executing ventilator therapy assessment process 248 or by multiple such devices in a distributed fashion. Procedure 500 begins at step 505 and continues on to step 510 where, as described in greater detail above, the device may receive one or more cardiorespiratory values and one or more other value regarding the subject undergoing ventilator therapy and/or the ventilator itself. In various embodiments, a cardiorespiratory value may be a measured blood pressure, heart rate, rate pressure product, or pulse pressure value. The one or more values may, in some embodiments, include a respiratory rate from the ventilator, a CO2 measurement, or a tidal volume value from the ventilator. For example, the CO2 measurement may be taken by an end tidal CO2 monitor, a transcutaneous monitor, or a blood gas analyzer, in various cases.

At step 515, the device selects a ventilation category (e.g., oxygenation/cardiovascular) based on the values received in step 510, as described in greater detail above. Notably, the ventilation category may be selected not only on the status of the ventilator itself and/or on the pulmonary effects on the subject, but also on the cardiovascular effects of the ventilator therapy on the subject. As detailed above in Table 1, each of the parameters from step 510 may have a corresponding threshold or expected range that has been either preset by default or set by a health care provider. The device may use these thresholds to define the triggering criteria for the different ventilation-related categories.

At step 520, the device retrieves an alert from memory based on the category selected in step 515, as detailed above. In some embodiments, the alert may indicate the category itself. In further embodiments, the alert may be a best practice alert that also includes a recommendation for a health care provider. For example, an alert associated with the “Tachypnea” category may suggest that the health care provider increase the tidal volume to a maximum acceptable value.

At step 525, the device provides the alert from step 520 to a user interface device, as described in greater detail above. For example, the alert may be provided to an electronic display or to a speaker, to notify a health care provider about the ventilation category. In some cases, the alert may be provided on a push basis (e.g., without first receiving a request). For example, an urgent alert may be sent directly to the interface device in response to identifying a certain ventilation category. In other cases, an alert may be non-urgent/information. In such cases, the device may provide the alert on a pull basis (e.g., the health care provider operates the interface device to request any informational alerts). Procedure 500 then ends at step 530.

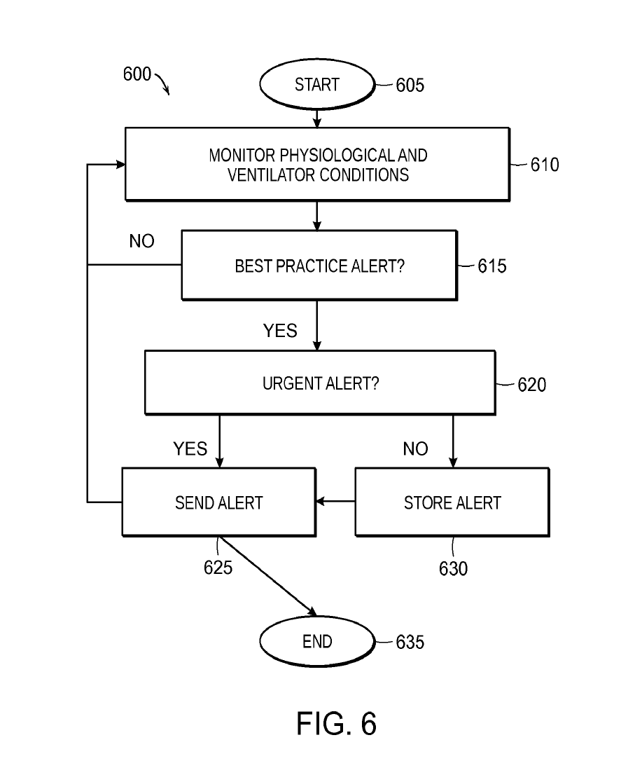

Referring now to FIG. 6, an example flow diagram of a procedure 600 is shown for selectively providing an alert regarding a subject undergoing respiratory therapy, according to various embodiments. Procedure 600 may be performed, for example, by device 200 executing process 248 or a combination of devices, in accordance with a distributed implementation. Procedure 600 may start at step 605 and continue on to step 610 where, as described in greater detail above, the device monitors the condition of the ventilator (e.g., parameter settings, associated measurements, etc.) and the physiological state of the subject undergoing therapy using the ventilator. In various embodiments, the device may use this data to classify the state of the subject (e.g., by applying a ventilation category, an oxygenation category, etc. to the subject).

At step 615, the device determines whether or not a best practice alert has been trigger, as detailed above. In particular, the device may determine whether or not a category assigned to the subject has an associated alert. For example, if the ventilation category of the subject is “Acceptable” and this category does not have an associated alert, procedure 600 may repeat steps 610-615 any number of times until such an alert is identified.

At step 620, the device determines whether or not the alert is an urgent alert, as detailed above. In some cases, the alerts associated with the categories applied by the device may have different degrees of urgency. For example, alerts for certain categories may be urgent, while alerts for other categories may be informational. If the alert is urgent, procedure 600 may continue on to step 625 where it sends the urgent alert to a user interface device. For example, the device may provide an urgent alert to a display screen, to notify a health care provider as to the current status/category of the subject. If the alert is not urgent, however, procedure may instead proceed to step 630 where the alert is stored in memory until a future point in time. At such a time, procedure 600 may proceed from step 630 to step 625 where the device sends the stored alert to the user interface device. For example, the device may send a stored informational alert to a display at some time in the future, such as when a health care provider requests all informational alerts. Procedure 600 then either ends at step 635 or, alternatively, may be repeated any number of times (e.g., to provide continuous monitoring and alerting functions regarding the subject).

It should be noted that while certain steps within procedures 500-600 may be optional as described above, the steps shown in FIGS. 5-6 are merely examples for illustration, and certain other steps may be included or excluded as desired. Further, while a particular order of the steps is shown, this ordering is merely illustrative, and any suitable arrangement of the steps may be utilized without departing from the scope of the embodiments herein. Moreover, while procedures 500-600 are described separately, certain steps from each procedure may be incorporated into each other procedure, and the procedures are not meant to be mutually exclusive.

Advantageously, the exemplary embodiments of the present invention allow data regarding a subject undergoing respiratory therapy using a ventilator to be aggregated and analyzed in a centralized manner. In some aspects, various categories are disclosed herein that quantify the ventilation, oxygenation, and other possible conditions that may be present during the therapy. Such categories provide a consistent framework to assess the effectiveness of the therapy and also take into account previously unconsidered factors, such as the cardiovascular reaction of the patient to the respiratory therapy. In further aspects, alerts associated with the categories may be retrieved and provided to a user interface device, thereby notifying the health care provider as to the state of the subject and/or recommending a course of action to the provider.

Experimental Results

An assessment utilizing the patient categorization system was conducted. In particular, the ventilation and oxygenation statuses of patients were categorized using near continuous data (e.g., five second sampling) in which the one minute median result were applied to the thirteen rules based algorithms. The targets were predetermined based on generally accepted values. The categories were defined as:
Acceptable ventilation=Target
Tachypnea=RR>Target for age
Severe tachypnea=RR>target for age & HR>target for age
Insufficient ventilation=ETCO₂>55 mmHg, Vt<4 mL/Kg, & HR>target for age
Hypoventilation=ETCO₂>55 mmHg
Severe hypoventilation=ETCO₂>55 mmHg, RR<target for age, & HR>target for age
Hyperventilation=ETCO₂<35 mmHg
Severe hyperventilation=ETCO₂<35, Vt>8 mL/Kg, RR>target for age, and HR<or> target for age
Hyperoxia=S/F>345
Normoxia=S/F 253-345
Mild hypoxemia=S/F 213-253
Moderate hypoxemia=S/F 153-212
Severe hypoxemia=S/F<153 for >

Furthermore the classifications were probabilistically summarized by calculating the % of time a subject belongs to a category. The experimental results included 60 patients with 9316 hrs of data. The median evaluation period was 74 hrs. The average oxygenation was within normoxia (86%) followed by: mild hypoxemia 8%; hyperoxia 4%; moderate hypoxemia 2%; and severe hypoxemia 0.3%. The average ventilation was acceptable 84% followed by: severe hyperventilation 7%; severe tachypnea 4%; hypoventilation 3%; hyperventilation 2%; and tachypnea 0.8%. The experimental results did not demonstrate any evidence of insufficient ventilation. The analysis demonstrated that patients can be successfully categorize based on the goals of therapy. Moreover, coupling categorization with machine learning, resulting in real-time decision support could improve quality.

While there have been shown and described illustrative embodiments that include specific categories, those skilled in the art will understand than there may be other ways to categorize the status of a subject undergoing respiratory therapy using a ventilator, thus the illustrative embodiment of the present invention should not be limited as such. For example, other embodiments may include different combinations of categories or different categories entirely, without deviating from the teachings herein. Furthermore, although some medical devices have been provided, the illustrative embodiment of the present invention can utilize data from any number of medical devices and may be displayed on any number of computerized devices, such as mobile phone, smartphone, computer, laptop computer, etc. Also, although the above technique has been described as being processed in a particular order, the illustrative embodiment is not necessarily limited as such since.

The foregoing description has been directed to specific embodiments. It will be apparent, however, that other variations and modifications may be made to the described embodiments, with the attainment of some or all of their advantages. For instance, it is expressly contemplated that the components and/or elements described herein can be implemented as software being stored on a tangible (non-transitory) computer-readable medium (e.g., disks/CDs/RAM/EEPROM/etc.) having program instructions executing on a computer, hardware, firmware, or a combination thereof. Accordingly this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A method for categorizing a state of a patient undergoing therapy from a mechanical ventilator, the method comprising:
receiving, at a device, one or more cardiovascular measurements regarding the subject, a respiratory rate from the ventilator, a carbon dioxide (CO2) measurement, and a tidal volume value from the ventilator;
selecting, by the device, a ventilation category based on the one or more cardiovascular measurements, the respiratory rate, the CO2 measurement, and the tidal volume value;
retrieving, by the device, an alert that is associated with the selected ventilation category; and
providing, by the device, the alert to a user interface device.

2. The method as in claim 1, wherein the one or more cardiovascular measurements comprise at least one of: a measured heart rate of the subject, a measured blood pressure of the subject, a rate pressure product value, or a pulse pressure value.

3. The method as in claim 1, wherein the ventilation category is selected from a set of categories comprising three or more of: an acceptable category, a hyperventilation category, a severe hyperventilation category, a hypoventilation category, a severe hypoventilation category, a tachypnea category, a severe tachypnea category, or an insufficient ventilation category.

4. The method as in claim 1, further comprising:
determining that the alert is an urgent alert, wherein the alert is provided to the user interface in response to determining that the alert is an urgent alert.

5. The method as in claim 1, further comprising: determining an oxygen saturation value;
selecting an oxygenation category based on the oxygen saturation value, wherein the oxygen saturation value comprises an oxygen saturation index (OSI) or an oxygen saturation to fraction of inspired oxygen (S/F) ratio associated with the subject;
retrieving an alert that is associated with the selected oxygenation category; and
providing the recommendation that is associated with the oxygenation category to the user interface device.

6. The method as in claim 5, wherein the oxygenation category is selected from a set of categories comprising: an acceptable category, a mild hypoxemia category, a moderate hypoxemia category, a severe hypoxemia category, or a hyperoxia category.

7. The method as in claim 6, further comprising:
analyzing a history of oxygenation categories associated with the subject to detect acute respiratory distress syndrome (ARDS) and, in response to detecting ARDS,
providing an ARDS alert to the user interface device.

8. The method as in claim 5, further comprising:
evaluating a history of subject measurements and operating conditions of the ventilator to detect a potential subject condition; and
reporting the detected subject condition to the user interface device.

9. The method as in claim 8, wherein the subject condition corresponds to at least one of: a ventilator associated condition being present in the subject, a ventilator associated lung injury being present in the subject, the subject requiring extubation readiness assessment, or the subject being ready for extubation.

10. The method as in claim 1, further comprising:
receiving, at the processor, the CO₂ measurement from an end tidal CO₂ monitor, a transcutaneous monitor, or a blood gas analyzer.

11. A system for categorizing a state of a subject undergoing therapy from a mechanical ventilator, comprising:
one or more network interfaces to communicate with a network;
a processor coupled to the one or more network interfaces and configured to execute one or more processes; and
a memory configured to store a process executable by the processor, the process when executed operable to:
receive one or more cardiovascular measurements regarding the subject, a
respiratory rate from the ventilator, a carbon dioxide (CO₂) measurement regarding the subject, and a tidal volume value from the ventilator;
select a ventilation category based on the one or more cardiovascular measurements, the respiratory rate, the CO₂ measurement, and the tidal volume value;
retrieve an alert that is associated with the selected ventilation category; and
provide the alert to a user interface device.

12. The system as in claim 11, wherein the one or more cardiovascular measurements comprise at least one of: a measured heart rate of the subject, a measured blood pressure of the subject, a rate pressure product value, or a pulse pressure value.

13. The system as in claim 11, wherein the ventilation category is selected from a set of categories comprising three or more of: an acceptable category, a hyperventilation category, a severe hyperventilation category, a hypoventilation category, a severe hypoventilation category, a tachypnea category, a severe tachypnea category, or an insufficient ventilation category.

14. The system as in claim 11, wherein the process when executed is further operable to:
determine that the alert is an urgent alert, wherein the alert is provided to the user interface in response to determining that the alert is an urgent alert.

15. The system as in claim 11, wherein the process when executed is further operable to: determine an oxygen saturation value;
select an oxygenation category based on the oxygen saturation value, wherein the oxygen saturation value comprises an oxygen saturation index (OSI) or an oxygen saturation to fraction of inspired oxygen (S/F) ratio associated with the subject;
retrieve an alert that is associated with the selected oxygenation category; and
provide the alert that is associated with the oxygenation category to the user interface device.

16. The system as in claim 15, wherein the oxygenation category is selected from a set of categories comprising: an acceptable category, a mild hypoxemia category, a moderate hypoxemia category, a severe hypoxemia category, or a hyperoxia category.

17. The system as in claim 16, wherein the process when executed is further operable to: analyze a history of at least 1 or more poor compliance, increased deadspace fraction, and/or mild, moderate or severe oxygenation categories associated with the subject to detect acute respiratory distress syndrome (ARDS) and, in response to detecting ARDS,
provide an ARDS alert to the user interface device.

18. The system as in claim 15, wherein the process when executed is further operable to:
evaluate a history of subject measurements and operating conditions of the ventilator to detect a potential subject condition; and
report the detected subject condition to the user interface device.

19. The system as in claim 18, wherein the subject condition corresponds to at least one of: a ventilator associated condition being present in the subject, a ventilator associated lung injury being present in the subject, the subject requiring extubation readiness assessment, or the subject being ready for extubation.

20. The system as in claim 11, wherein the process when executed is further operable to:
receive the CO₂ measurement from an end tidal CO₂ monitor, a transcutaneous monitor, or a blood gas analyzer.

21. A non-transitory computer readable medium containing program instructions executed by a processor, the programming instructions comprising:
program instructions that receive one or more cardiovascular measurements regarding the subject, a respiratory rate from the ventilator, a carbon dioxide (CO₂) measurement regarding the subject, and a tidal volume value from the ventilator;
program instructions that select a ventilation category based on the one or more cardiovascular measurements, the respiratory rate, the CO₂ measurement, and the tidal volume value;
program instructions that retrieve an alert that is associated with the selected ventilation category; and
program instructions that provide the alert to a user interface device.

22. The method as in claim 1, wherein the one or more respiratory measurements comprise at least one of: the respiratory rate, the tidal volume value, a fraction of inspired oxygen value (FIO2), a compliance measurement, a resistance measurement, a CO2 measurement, or the VO2 measurement (oxygen consumption).

23. The method as in claim 1, further comprising:
receiving, at the processor, the oxygen measurement from a pulse oximeter, a transcutaneous monitor or a blood gas analyzer.

24. The system as in claim 11, wherein the process when executed is further operable to: determine an oxygen measurement value;
select an oxygenation category based on the oxygen measurement value, wherein the oxygen measurement value comprises a measurement from an pulse oximeter value (SpO2) or a transcutaneous monitor or a blood gas analyzer associated with the subject;
retrieve an alert that is associated with the selected oxygenation category; and
provide the alert that is associated with the oxygenation category to the user interface device.
